(19) United States
(12) Patent Application Publication
(10) Pub. No.: US 2017/0143702 A1
(43) Pub. Date: May 25, 2017

(54) Treatment of Motor Neuronopathies

(71) Applicant: CENTRE NATIONAL DE LA RECHERCHE SCIENTIFIQUE- CNRS, Paris (FR)

(72) Inventors: Suzie Lefebvre, Paris (FR); Kevinee Khoobarry, Paris (FR); Philippe Burlet, Paris (FR)

(73) Assignee: CENTRE NATIONAL DE LA RECHERCHE SCIENTIFIQUE- CNRS, Paris (FR)

(21) Appl. No.: 15/426,586

(22) Filed: Feb. 7, 2017

Related U.S. Application Data
(63) Continuation of application No. 14/429,325, filed on Mar. 18, 2015.

(30) Foreign Application Priority Data
Sep. 19, 2012 (FR) 12 58796

(51) Int. Cl. A61K 31/495; A61K 45/06; A61K 31/197; A61K 31/343

(52) U.S. Cl. CPC A61K 31/495; A61K 31/343; A61K 45/06; A61K 31/197

(57) ABSTRACT

The invention relates to a calcium channel blocker selected from the group of the calcium channel blockers of the phenylalkylamine family, the calcium channel blockers of the amino acid family, and the calcium channel blockers of the benzofuran family, for the use thereof in the treatment of a motor neuronopathy.

The invention relates to a calcium channel blocker selected from the group of calcium channel blockers of the phenylalkylamine family, calcium channel blockers of the amino acid family and calcium channel blockers of the benzofuran family, for use thereof in the treatment of a motor neuronopathy.

Spinal muscular atrophy (SMA) is a neuromuscular disease transmitted in an autosomal recessive manner. It is characterized by a degeneration of the alpha motor neurons from the anterior horn of the spinal cord leading to muscular atrophy and resulting in paralysis. This alpha motor neuron degeneration thus substantially compromises the vital prognosis of patients. In healthy subjects, these neurons transmit messages from the brain to the muscles, leading to the contraction of the latter. In the absence of such a stimulation, the muscles atrophy. Subsequently, in addition to a generalized weakness and atrophy of the muscles, and more particularly of those of the trunk, upper arms and thighs, these disorders can be accompanied by serious respiratory problems.

There is a strong correlation between the age at which the symptoms appear and the severity of the condition, such that, the earlier the disease begins, the less favorable the vital prognosis. It is according to this criterion that this disease has been categorized as three clinical types as follows:
type I (Werdnig-Hoffman disease), characterized by an early onset, generally before six months old, and in which afflicted children are unable to sit assisted and progression is severe. The life expectancy of affected children rarely exceeds three years and is often limited to a few months;
type II or intermediate form of Werdnig-Hoffman disease, which usually appears between six months and three years old, in which affected children can sit unassisted but are never able to walk unaided. This clinical type is often associated with frequent respiratory infections which can complicate the cause of this condition and reduce life expectancy;
type III or Kugelberg-Welander disease, which generally appears around three-four years old and sometimes up to 21 years old and in which affected individuals can walk but have more or less pronounced problems with walking depending on the severity, which is very variable from one patient to another. This type of the disease generally does not compromise life expectancy.

All the forms of spinal muscular atrophy are accompanied by progressive muscle weakness and atrophy subsequent to the degeneration of the neurons from the anterior horn of the spinal cord. SMA currently constitutes one of the most common causes of infant mortality. It equally affects girls or boys in all regions of the world with a prevalence of between 1/6000 and 1/10 000.

The gene involved in spinal muscular atrophies has been located on chromosome 5 in position q12-q13, whatever the clinical type that presents. This gene encodes the survival motor neuron (SMN) protein. This protein is located in the cytoplasm and the nucleus, where it accumulates in punctate domains called Cajal bodies (CBs). A defect in this accumulation is described in SMA.

It is accepted that SMA is linked to an inactivation of the SMN gene. More specifically, two genes encoding the SMN protein: SMN1 and SMN2, both genes being transcribed, have been brought to light. The analysis of their promoters has shown that these elements are virtually identical both at their sequence level and at their activity level. Thus, the SMN2 gene encodes the same SMN protein, but in a lesser amount. It has, moreover, been noted that the inactivation of a SMN1 can be partly overcome by the expression of the virtually identical SMN2 gene.

Medical or paramedical care is currently available to patients suffering from SMA, providing them with the best possible quality of life, such as:

motor physiotherapy and hydrotherapy which consist in helping patients to form their body scheme;

respiratory physiotherapy which makes it possible to relieve bronchial congestion in patients, thus reducing the risk of respiratory ailments; or else

orthopedic treatment to prevent deformations of the skeleton and of the joints.

However, this care is aimed only at relieving symptoms and preventing the complications of the disease. It does not make it possible to treat the pathological condition.

Furthermore, amyotrophic lateral sclerosis (ALS) or Charcot disease is a neurodegenerative disease which affects mainly adults, and which is characterized by weakening and then paralysis of the leg and arm muscles, of the respiratory muscles and also of the swallowing and speech muscles. The prevalence of this disease is estimated at one patient out of 25 000 individuals.

This disease also involves degeneration of the motor neurons, in particular those of the cerebral cortex and of the anterior horn of the spinal cord. This degeneration results in a destruction of the pyramidal tract. ALS can present as two main forms: the “spinal” form and the “bulbar” form. The spinal form is due to the degeneration of the motor neurons located in the spinal cord, while the bulbar form corresponds to the degeneration of the motor neurons of the spinal bulb. These two forms can follow one another or can develop simultaneously, the disease almost always progressing to a complete (spinal and bulbar) form.

Patients suffering from ALS have available to them medical care aimed not at the recovery of the motor functions, but at the maintaining of the remaining functions, in particular by means of physiotherapy and speech therapy. In addition, a therapeutic strategy based on Riluzole (sold under the name Rilutek®) has recently been proposed. However, this strategy only makes it possible to effectively delay the progression of the pathological condition and prolongs the phase of the disease during which the patient is independent. Also, this compound improves patient prognosis by only 30%, which remains unsatisfactory. There is currently no effective treatment against the appearance and/or the development of ALS.

There is no treatment for the purpose of effectively stopping the progression of motor neuronopathies, in particular for curbing the degeneration of motor neurons. There is therefore a longstanding need for an effective therapeutic strategy against motor neuronopathies, in particular motor neuronopathies of spinal muscular atrophy and amyotrophic lateral sclerosis type.

The inventors have now discovered that calcium channel blockers of the phenylalkylamine, amino acid and benzofuran family make it possible, on the one hand, to significantly increase the distribution of SMN in CBs and, on the other hand, to improve a defect in snRNP accumulation in CBs. Consequently, these calcium channel inhibitors constitute a relevant therapeutic strategy in the treatment of motor neuronopathies, in particular SMA or ALS.

The invention therefore relates to a calcium channel blocker selected from the group consisting:
of calcium channel blockers of the phenylalkylamine family of formula I and salts thereof,

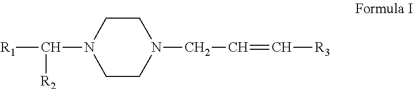

in which:
   the R₁, R₂ and R₃ groups are identical or different and are selected from phenyl and fluorophenyl radicals, provided that at least one of the R₁, R₂ and R₃ groups is a fluorophenyl radical;
of calcium channel blockers of the amino acid family of formula II,

H₂N—CH₂—C(R₄)R′₄—CH₂—COOR₅ Formula II
in which:
   the R₄ group is a linear- or branched-chain lower alkyl radical containing up to eight carbon atoms and the R′₄ group is a hydrogen atom,
      or
      R₄ and R′₄ together form a cyclic lower alkyl radical containing up to eight carbon atoms, and
   the R₅ group is a hydrogen atom or a linear- or branched-chain lower alkyl radical containing up to eight carbon atoms;
of calcium channel blockers of the benzofuran family of formula III and salts thereof,

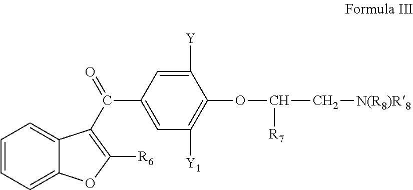

in which:
   the R₆ group represents a linear- or branched-chain lower alkyl radical containing up to six carbon atoms,
   the R₇ group represents a hydrogen atom or a methyl group,
   the N(R₈)R′₈ group represents a dimethylamino, diethylamino, dipropylamino, piperidino, pyrrolidino or morpholino group, and
   the Y and Y₁ groups are identical and represent a hydrogen, iodine or bromine atom;
      for use thereof in the treatment of a motor neuronopathy selected from spinal muscular atrophy, amyotrophic lateral sclerosis, primary lateral sclerosis, Kennedy's disease and Charcot-Marie-Tooth disease.

The term “motor neurons” is intended to mean the neurons constituting the path of exit from the central nervous system (or final path) of any motor action. The cell bodies of motor neurons are located either in the brainstem, or in the ventral horn of the grey matter of the spinal cord. Each motor neuron has an axon which leaves the central nervous system so as to innervate the muscle fibers of a muscle. The assembly made up of a motor neuron and the muscle fibers that it innervates constitutes a motor unit. Three types of motor neurons are distinguished: “alpha motor neurons”, which innervate the muscle fibers responsible for contraction, “gamma motor neurons”, which innervate neuromuscular bundles, thus adjusting their sensitivity to stretching, and also “beta motor neurons”, which innervate both types of fibers. Preferentially, the compound of the invention is particularly advantageous for the treatment of motor neuronopathies involving alpha motor neuron degeneration.

The term “motor neuronopathy”, is intended to mean a disease involving motor neuron degeneration, which manifests itself through an absence of muscle stimulation resulting in amyotrophy.

This disease is associated with an SMN protein deficiency and/or in a reduction in the number of CBs and/or a defect in localization of the SMN protein to CBs.

The symptoms of such a pathological condition may be varied and may comprise:
a progressive motor deficiency;
amyotrophy;
fasciculations; and/or
cramps.

Said motor neuronopathy is spinal muscular atrophy, amyotrophic lateral sclerosis, primary lateral sclerosis, Kennedy's disease, or Charcot-Marie-Tooth disease. Preferentially, said motor neuronopathy is spinal muscular atrophy or amyotrophic lateral sclerosis. More preferentially, said neuronopathy is spinal muscular atrophy. Typically, said spinal muscular atrophy is of type I, of type II or of type III.

The term “calcium channel blocker” or “calcium channel inhibitor” is intended to mean a compound which inhibits the entry of calcium into cells via voltage-dependent calcium channels. This therapeutic class is particularly well documented in the prior art, in particular for use thereof in cardiology, in particular in the treatment of arterial hypertension, angina or alternatively paroxysmal junctional tachycardia attacks.

These calcium channel blockers are described, inter alia, by Théophile Godfraind in the manual Calcium Channel Blockers, published by the publications Milestones in Drug Therapy on Mar. 26, 2004.

The inventors have now brought to light a new role for these calcium channel blockers. Indeed, they have shown that the latter allow an increase in the distribution of the SMN protein and of snRNPs to Cajal bodies and/or an increase in the number of Cajal bodies.

Thus, the calcium channel blockers of the invention, by significantly increasing the distribution of the SMN protein and of snRNPs to Cajal bodies and/or the number of Cajal bodies, constitute a relevant therapeutic strategy for the treatment of motor neuronopathies, in particular SMA.

The term “Cajal bodies” or alternatively “CBs” is intended to mean the site of the initial modifications and of the assembly of several small nuclear RNAs with their ribonucleoproteins, snRNPs (small nuclear ribonucleoproteins) imported from the cytoplasm, or recycled to the nucleus. CBs have an ultrastructure which reveals coiled threads, hence their other name of “coiled bodies”. CBs are dynamic structures and their number can vary rapidly if there is a change in the level of transcription. The abundance of CBs can be easily determined by those skilled in the art, in particular by immunodetection of the localization of snRNPs to CBs and the number of CBs thus detected, or more specifically by detection of the coilin protein in CBs. As previously indicated, the SMN protein accumulates in Cajal bodies. A defect in this accumulation is, moreover, described in SMA.

The inventors have also demonstrated the fact that the calcium channel blockers of the invention allow an increase in snRNPs at the level of Cajal bodies.

The SMN protein is part of a broad multiprotein complex, the role of which is the assembly of splicing ribonucleoprotein particles, snRNPs. A defect in this assembly is visualized by a marked decrease in the localization of snRNPs at the level of the CBs in the fibroblasts of SMA patients. Thus, the calcium channel blockers of the invention, by increasing this accumulation of snRNPs at the level of CBs, make it possible to effectively treat motor neuronopathies, in particular SMA.

### Calcium Channel Blocker of the Phenylalkylamine Family

Typically, the calcium channel blocker of the invention may be a blocker of the phenylalkylamine family of formula I or a salt thereof:

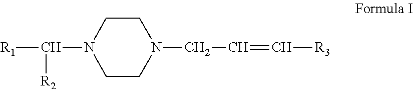

in which:
   the R₁, R₂ and R₃ groups are identical or different and are selected from phenyl and fluorophenyl radicals, provided that at least one of the R₁, R₂ and R₃ groups is a fluorophenyl radical.

This class of calcium channel blockers is described by Théophile Godfraind in the manual Calcium Channel Blockers, published by the publications Milestones in Drug Therapy on Mar. 26, 2004. These calcium channel blockers of formula I are also described in application FR 2 014 487 (Janssen Pharmaceutica). Formula I of the invention encompasses all the Z or E geometric stereoisomers. Preferentially, formula I of the invention is in the E configuration.

In one preferred embodiment:
the R₁ and R₂ groups are para-fluorophenyl radicals; and
the R₃ group is a phenyl radical.

In this embodiment, said calcium channel blocker of the phenylalkylamine family is flunarizine or a salt thereof. It is preferably flunarizine dihydrochloride. Thus, the invention relates to flunarizine or a salt thereof, for use thereof in the treatment of a motor neuronopathy selected from spinal muscular atrophy, amyotrophic lateral sclerosis, primary lateral sclerosis, Kennedy's disease and Charcot-Marie-Tooth disease.

Indeed, the inventors have shown that this compound provides the best results with regard to increasing the accumulation of the SMN protein and/or of snRNPs in CBs. Typically, the flunarizine dihydrochloride can be administered at a dose of between 5 mg and 10 mg per day.

Flunarizine has the formula C₂₆H₂₈Cl₂F₂N₂ as represented in formula Ia:

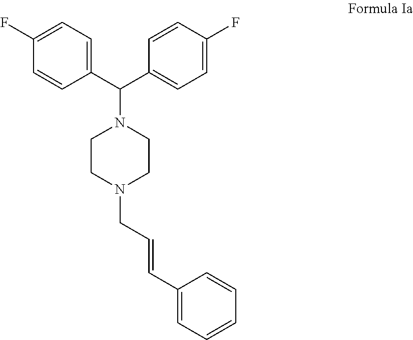 [SMILES: Fc1ccc(C(c2ccc(F)cc2)N2CCN(C/C=C/c3ccccc3)CC2)cc1]

Flunarizine bears the CAS number: 30484-77-6. This molecule is known for its use in the treatment of migraines and is sold under the name Sibelium®. It is an inhibitor of the calcium channels of neurons and of the calcium channels of blood vessels, used in the treatment of dizziness of vestibular origin and of migraine.

### Calcium Channel Blockers of the Amino Acid Family

Typically, the calcium channel blocker of the invention may be a calcium channel blocker of the amino acid family of formula II:

H₂N—CH₂—C(R₄)R′₄—CH₂—COOR₅ Formula II
in which:
   the R₄ group is a linear- or branched-chain lower alkyl radical containing up to eight carbon atoms and the R′₄ group is a hydrogen atom,
      or
      R₄ and R′₄ together form a cyclic lower alkyl radical containing up to eight carbon atoms, and
   the R₅ group is a hydrogen atom or a linear- or branched-chain lower alkyl radical containing up to eight carbon atoms.

Typically, the lower alkyl radicals R₄ or R₅ of formula II are alkyl radicals containing from one to four carbon atoms, in particular a methyl, ethyl, isopropyl, isobutyl or tert-butyl radical.

This category of calcium channel blockers is described by Théophile Godfraind in the manual Calcium Channel Blockers, published by the publications Milestones in Drug Therapy on Mar. 26, 2004. These calcium channel blockers of formula (II) are also described in application FR 2 294 697 (Warner Lambert Company).

In one preferred embodiment:
R₄ and R′₄ together form a cyclohexane group, and
the R₅ group is a hydrogen atom.

In this preferred embodiment, said calcium channel blocker of the amino acid family is gabapentin. Thus, the invention relates to gabapentin for use thereof in the treatment of a motor neuronopathy selected from spinal muscular atrophy, amyotrophic lateral sclerosis, primary lateral sclerosis, Kennedy's disease and Charcot-Marie-Tooth disease.

Gabapentin or 2-[1-(aminomethyl)cyclohexyl]acetic acid has the empirical formula C₉H₁₇NO₂ and is represented by formula (IIa).

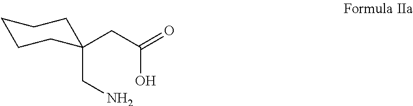 [SMILES: NCC1(CC(=O)O)CCCCC1]

Gabapentin bears the CAS number: 60142-96-3. It is known for its antiepileptic activity. It is, moreover, sold under the name Neurontin®.

In another embodiment,
the R₄ group is an isobutyl, and
the R′₄ and R₅ groups are a hydrogen atom.

In this particular embodiment, said calcium channel blocker of the amino acid family is pregabalin. Thus, the invention relates to pregabalin for use thereof in the treatment of a motor neuronopathy selected from spinal muscular atrophy, amyotrophic lateral sclerosis, primary lateral sclerosis, Kennedy's disease and Charcot-Marie-Tooth disease.

Pregabalin has the empirical formula C₈H₁₇NO₂ and is represented in formula IIb:

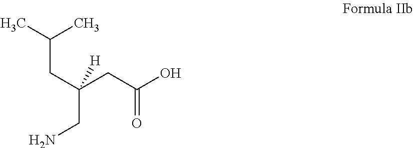

Pregabalin bears the CAS number 148553-50-8. It is known for its antiepileptic activity.

### Calcium Channel Blockers of the Benzofuran Family

Typically, the calcium channel blocker of the invention may be a blocker of the benzofuran family of formula III or a salt thereof:

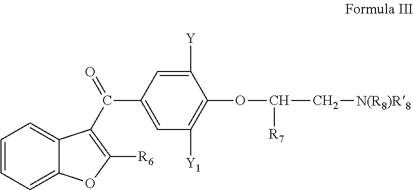

in which:
   the R₆ group represents a linear- or branched-chain lower alkyl radical containing up to six carbon atoms,
   the R₇ group represents a hydrogen atom or a methyl group,
   the N(R₈)R′₈ group represents a dimethylamino, diethylamino, dipropylamino, piperidino, pyrrolidino or morpholino group, and
   the Y and Y₁ groups are identical and represent a hydrogen, iodine or bromine atom.

This category of calcium channel blockers is described by Théophile Godfraind in the manual Calcium Channel Blockers, published by the publications Milestones in Drug Therapy on Mar. 26, 2004. These calcium channel blockers of formula (III) are also described in application FR 1 339 389 (Société belge de l'azote et des produits chimiques du Marly).

In one preferred embodiment,
the R₆ group is a butyl,
the R₇ group is a hydrogen atom,
the N(R₈)R′₈ group is a diethylamino group, and
the Y and Y₁ groups are identical and are an iodine atom.

In this embodiment, the blocker of the benzofuran family is amiodarone or a salt thereof. Thus, the invention relates to amiodarone or a salt thereof, for use thereof in the treatment of a motor neuronopathy selected from spinal muscular atrophy, amyotrophic lateral sclerosis, primary lateral sclerosis, Kennedy's disease and Charcot-Marie-Tooth disease.

Amiodarone or (2-butyl-3-benzofuranyl)[4-[2-(diethylamino)ethoxy]-3,5-diiodophenyl]methanone hydrochloride is a compound of empirical formula C₂₅H₂₉I₂NO₃ and is represented by formula Ma.

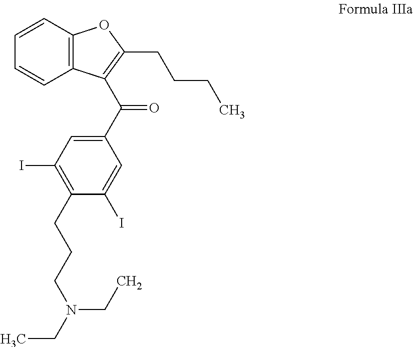 [SMILES: CCCCc1oc2ccccc2c1C(=O)c1cc(I)c(CCCN(CC)CC)c(I)c1]

Amiodarone bears the CAS number: 96027-74-6. It is known for its antiarrythmic activity.

### Method of Treatment

The invention also relates to methods for treating a subject suffering from a motor neuronopathy, such as spinal muscular atrophy, amyotrophic lateral sclerosis, primary lateral sclerosis, Kennedy's disease or else Charcot-Marie-Tooth disease, comprising the step of administering, to said subject, a therapeutically effective amount of at least the calcium channel blockers of the invention. The term “therapeutically effective amount” is intended to mean an amount sufficient for treating and/or treating or stopping the progression of motor neuronopathies.

### Pharmaceutical Compositions

The calcium channel blockers of the invention may also be used for preparing pharmaceutical compositions for the treatment of motor neuronopathies such as spinal muscular atrophy, amyotrophic lateral sclerosis, primary lateral sclerosis, Kennedy's disease or else Charcot-Marie-Tooth disease. Thus, the invention relates to pharmaceutical compositions for use in methods for treating motor neuronopathies in humans, said compositions comprising at least one calcium channel blocker according to the invention and a pharmaceutically acceptable carrier.

The calcium channel blockers of the invention are known and are available on the market. It will be possible to use the formulations already developed and employed for each of the calcium channel blockers of the invention. Any calcium channel blocker according to the invention may be combined with any type of pharmaceutically acceptable carrier or excipient, in particular those already described in the prior art for use thereof for the calcium channel blockers of the invention, in order to form a pharmaceutical composition according to the invention. The term “pharmaceutically acceptable” refers to molecular entities and compositions which do not produce an opposite reaction, allergic reaction or other unwanted reaction when they are administered to a mammal, particularly a human being. A pharmaceutically acceptable carrier or excipient may be solid, semi-solid or liquid.

The form of the pharmaceutical compositions, their route of administration, their dosage and their dosage regimen naturally depend on the severity of the pathological condition, on its stage of progression, on the age, sex and weight of the subject to be treated, etc. Those skilled in the art will therefore take care to adjust the dosages according to the patient to be treated, in particular during the treatment of SMA in which the patients are children.

The pharmaceutical compositions according to the invention can be formulated for topical, oral, systemic, intranasal, parenteral, intravenous, intramuscular, subcutaneous administration, or the like. According to the mode of administration, the composition comprising a calcium channel blocker according to the invention may be in any of the pharmaceutical forms.

Preferably, the calcium channel blocker according to the invention is included in a composition which is administered orally. For oral administration, the compositions may be in the form of tablets, gel capsules, sugar-coated tablets, syrups, suspensions, solutions, powders, granules, emulsions, or lipid or polymneric microspheres or nanospheres or vesicles for controlled release.

For topical application to the skin, the composition may have the form in particular of an aqueous or oily solution or of a dispersion of the lotion of serum type; of an emulsion of liquid or semi-liquid consistency of the milk type, obtained by dispersion of a fatty phase in an aqueous phase (O/W) or vice versa (W/O); of an emulsion of soft consistency of the cream type; of a two-phase emulsion; of an aqueous or anhydrous gel; of a foam or else of microcapsules or microparticles, or of vesicular dispersions of ionic and/or nonionic type, or else of spray formulas. These compositions are prepared according to the usual methods known to those skilled in the art.

For systemic application, the composition may be in the form of an aqueous or oily solution or in the form of a serum.

In one preferred embodiment, the composition according to the invention comprises a calcium channel blocker of the phenylalkylamine family according to the invention and a calcium channel blocker of the benzofuran family according to the invention.

Thus, the invention also relates to a composition comprising:
a calcium channel blocker of the phenylalkylamine family of formula I and salts thereof,

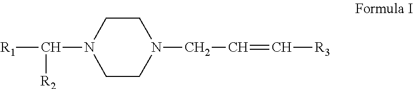

in which:
   the R₁, R₂ and R₃ groups are identical or different and are selected from phenyl and fluorophenyl radicals, provided that at least one of the R₁, R₂ and R₃ groups is a fluorophenyl radical;
and
a calcium channel blocker of the benzofuran family of formula III and salts thereof,

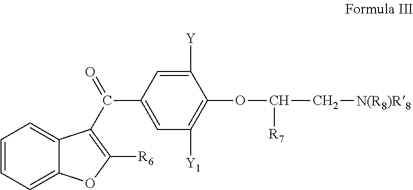

in which:
   the R₆ group represents a linear- or branched-chain lower alkyl radical containing up to six carbon atoms,
   the R₇ group represents a hydrogen atom or a methyl group,
   the N(R₈)R′₈ group represents a dimethylamino, diethylamino, dipropylamino, piperidino, pyrrolidino or morpholino group, and
   the Y and Y₁ groups are identical and represent a hydrogen, iodine or bromine atom;
      for use thereof in the treatment of a motor neuronopathy selected from spinal muscular atrophy, amyotrophic lateral sclerosis, primary lateral sclerosis, Kennedy's disease and Charcot-Marie-Tooth disease.

All the technical characteristics mentioned above apply here.

Preferentially, said calcium channel blocker of the phenylalkylamine family of formula I is flunarizine, and more preferentially flunarizine dihydrochloride. Preferentially, said calcium channel blocker of the benzofuran family of formula III is amiodarone hydrochloride.

The inventors have in fact shown that the combined administration of flunarizine dihydrochloride and amiodarone hydrochloride exerts a synergistic effect, which goes beyond the simple accumulation of the effects of said two compounds taken separately.

Thus, flunarizine dihydrochloride and amiodarone hydrochloride act in synergy on the distribution of the SMN protein to CBs in patient fibroblasts.

Consequently, the invention also relates to a composition comprising flunarizine dihydrochloride and amiodarone hydrochloride, for use thereof in the treatment of a motor neuronopathy selected from spinal muscular atrophy, amyotrophic lateral sclerosis, primary lateral sclerosis, Kennedy's disease and Charcot-Marie-Tooth disease.

In this particular embodiment, the combination of flunarizine dihydrochloride and amiodarone hydrochloride is administered at a dose of less than 5 mg per day. By substantially reducing the dose of active ingredient administered for treating a patient suffering from a motor neuronopathy, the inventors have developed a therapeutic strategy which considerably reduces the risk of occurrence of harmful and/or side effects linked to the treatment. Such a therapeutic strategy therefore proves to be particularly relevant, in particular in the chronic treatment of young patients suffering from motor neuronopathy.

In another embodiment, the composition according to the invention may combine the calcium channel blockers according to the invention with other active agents, in particular active agents capable of treating motor neuronopathies. Among these active agents, mention may be made, by way of example, of:
riluzole, (Haddad H et al. Riluzole attenuates spinal muscular atrophy disease progression in a mouse model, 2003. 28(4):432-437; and Wadman R I et al. 2012. Drug treatment for spinal muscular atrophy type I, Cochrane Database Syst Rev. 4:CD006281);
hydroxyurea (Chen T H et al, Randomized, double-blind, placebo-controlled trial of hydroxyurea in spinal muscular atrophy, Neurology, 2010 75:2190-21);
ubiquitin/proteasome inhibitors (Chang H C et al., Degradation of survival motor neuron (SMN) protein is mediated via the ubiquitin/proteasome pathway, Neurochem Int., 2004, 45:1107-1112; and Kwon D Y et al., Increasing expression and decreasing degradation of SMN ameliorate the spinal muscular atrophy phenotype in mice, Hum Mol Genet., 2011, 20:3667-3677);
histone deacetylase inhibitors (Lunke S et al., The emerging role of epigenetic modifications and chromatin remodeling in spinal muscular atrophy. J Neurochem, 2009, 109(6):1557-1569);
protein phosphatase inhibitors (Novoyatleva T et al., Protein phosphatase 1 binds to the RNA recognition motif of several splicing factors and regulates alternative pre-mRNA processing, Hum. Mol. Genet., 2008, 17, 52-70);
olesoxime (TRO19622), (Bordet T et al., Identification and characterization of cholest-4-en-3-one, oxime (TRO19622), a novel drug candidate for amyotrophic lateral sclerosis, J Pharmacol Exp Ther, 2007, 322(2):709-720);
aminoglycosides (Mattis V B et al., Analysis of a read-through promoting compound in a severe mouse model of spinal muscular atrophy, 2012, 525(1):72-75);
salbutamol (Pane M et al., Daily salbutamol in young patients with SMA type II, Neuromuscul Disord., 2008, 18:536-540);
isoindoline (application US 2010/0267712);
ibudilast (application US 2009/0062330);
kinase modulators (Burnett B G, et al., Regulation of SMN protein stability, Mol Cell Biol., 2009, 29(5):1107-1115);
IGF-1 variants (Murdocca M et al., IPLEX administration improves motor neuron survival and ameliorates motor functions in a severe mouse model of SMA, Mol Med. 2012 May 29. doi: 10.2119/molmed.2012.00056);
human growth factors (application US 2008/0187512);
PTK-inhibiting bicyclic pyrimidines (Hastings M L et al., Tetracyclines that promote SMN2 exon 7 splicing as therapeutics for spinal muscular atrophy, Sci Transl Med., 2009, 1(5):5ra12);
EGFR-inhibiting quinazoline (Butchbach M E et al., Effects of 2,4-diaminoquinazoline derivatives on SMN expression and phenotype in a mouse model for spinal muscular atrophy, Hum Mol Genet., 2010, 19(3):454-467);
tyrosine kinase inhibitors (application US 2010/0305036);
VEGFR inhibitors (application US 2010/0331296);
HSP90 inhibitors (Suzuki K et al., Pathogenesis-targeting therapeutics for spinal and bulbar muscular atrophy (SBMA), Neuropathology, 2009, 29(4):509-516);
myostatin antagonists (Rose F F Jr et al., Delivery of recombinant follistatin lessens disease severity in a mouse model of spinal muscular atrophy, Hum Mol Genet., 2009, 18(6):997-1005);
low-molecular-weight heparin (application US 2002/0040013);
neramexane (application US 2010/0234402);
nicergoline (application US 2003/0134869);
inhibitors of protein kinase G3SKB (Makhortova N R et al., A screen for regulators of survival of motor neuron protein levels, Nat Chem Biol., 2011, 7(8):544-552);
inhibitors of RhoA and of Rho-associated kinase (ROCK pathway) (Bowerman M et al., Mol Cell Neurosci, 2009, 42:66-74 and Bowerman M et al., Hum Mol Genet 2010, 19: 1468-1478);
antioxidants (Wan L et al., Inactivation of the SMN complex by oxidative stress, Mol Cell., 2008 31(2):244-254);
apoptosis inhibitors (Sareen D et al., Inhibition of apoptosis blocks human motor neuron cell death in a stem cell model of spinal muscular atrophy. PLoS One. 7(6):e39113. 2012); or else
ERK and PI3K/AKT kinase pathway modulators (Biondi O et al. In vivo NMDA receptor activation accelerates motor unit maturation, protects spinal motor neurons, and enhances SMN2 gene expression in severe spinal muscular atrophy mice, J Neurosci., 2010 30:11288-11299).

Thus, the invention also relates to a composition comprising at least one calcium channel blocker selected from the group consisting:
of calcium channel blockers of the phenylalkylamine family of formula I and salts thereof,

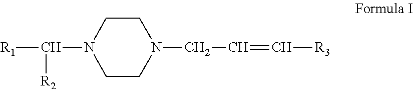

in which:
   the R₁, R₂ and R₃ groups are identical or different and are selected from phenyl and fluorophenyl radicals, provided that at least one of the R₁, R₂ and R₃ groups is a fluorophenyl radical;
of calcium channel blockers of the amino acid family of formula II,

H₂N—CH₂—C(R₄)R′₄—CH₂—COOR₅ Formula II
in which:
   the R₄ group is a linear- or branched-chain lower alkyl radical containing up to eight carbon atoms and the R′₄ group is a hydrogen atom,
      or
      R₄ and R′₄ together form a cyclic lower alkyl radical containing up to eight carbon atoms, and
   the R₅ group is a hydrogen atom or a linear- or branched-chain lower alkyl radical containing up to eight carbon atoms;
of calcium channel blockers of the benzofuran family of formula III and salts thereof,

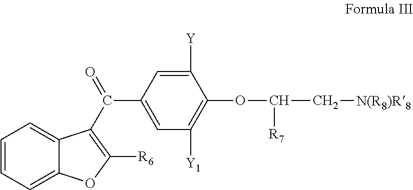

in which:
   the R₆ group represents a linear- or branched-chain lower alkyl radical containing up to six carbon atoms,
   the R₇ group represents a hydrogen atom or a methyl group,
   the N(R₈)R′₈ group represents a dimethylamino, diethylamino, dipropylamino, piperidino, pyrrolidino or morpholino group, and
   the Y and Y₁ groups are identical and represent a hydrogen, iodine or bromine atom;

and

at least one active agent selected from riluzole, hydroxyurea, ubiquitin/proteasome inhibitors, histone deacetylase inhibitors, protein phosphatase inhibitors, olesoxime, aminoglycosides, salbutamol, isoindoline, ibudilast, kinase modulators, IGF-1 variants, human growth factors, PTK-inhibiting bicyclic pyrimidines, EGFR-inhibiting quinazoline, tyrosine kinase inhibitors, HSP90 inhibitors, myostatine antagonists, low-molecular-weight heparin, neramexane, inhibitors of the protein kinase G3 SKB, inhibitors of RhoA and of Rho-associated kinase (ROCK pathway), antioxidants, apoptosis inhibitors and ERK and PI3K/AKT kinase pathway modulators.

Preferentially, said calcium channel blocker is flunarizine or a salt thereof.

Typically, the combining of the calcium channel blockers of the invention and of said active agent may be simultaneous, separate or spread out over time.

Typically, the calcium channel blockers of the invention may be combined with Riluzole for use thereof in the treatment of amyotrophic lateral sclerosis.

All the references cited in this application are incorporated by way of reference.

### FIGURES

FIG. 1: Effect of synergy between flunarizine dihydrochloride and amiodarone hydrochloride on the distribution of the SMN protein to CBs.

This figure shows the effects on immortalized SMA fibroblasts:
of DMSO (control for which a value of induction by a factor equal to 1 was arbitrarily assigned),
of flunarizine dihydrochloride alone,
of amiodarone hydrochloride alone, and
of the combination of flunarizine dihydrochloride and amiodarone hydrochloride.

### EXAMPLES

### 1. Test of the Calcium Channel Blockers of the Invention on Immortalized SMA Fibroblasts

The inventors have shown a beneficial effect of calcium channel blockers of the phenylalkylamine family, of the amino acid family and of the benzofuran family on the number of Cajal bodies of immortalized SMA fibroblasts.

To do this, they tested characteristic molecules each representing the best of these families:
flunarizine dihydrochloride,
gabapentin,
pregabalin, and
amiodarone hydrochloride.

The immortalization, using an SV40 virus T antigen, of fibroblasts isolated from a child suffering from a severe form of the disease made it possible to establish particularly robust cell culture conditions. This immortalization is described in the publication Lefebvre S, Burlet P, Viollet L, Bertrandy S, Huber C, Belser C and Munnich A. A novel association of the SMN protein with major non-ribosomal nucleolar proteins and its implication in spinal muscular atrophy. Hum Mol Genet (2002) 11 (9): 1017-1027.

The increase in the number of CBs in the immortalized SMA fibroblasts (10 000 to 12 000 cells per 0.8 to 1 cm squared well in a controlled culture medium) was evaluated after 24 hours of treatment for each of the calcium channel blockers at a concentration of 2 micrograms/ml (i.e. between 2 and 10 micromolar, depending on the molecular weight of the substance). The blockers of the invention are commercially available (for example from Sigma-Aldrich) and are sold in 96-well plates, at 2 milligrams per molecule per well to be dissolved in 1 ml of DMSO.

The concentration of 2 micrograms/ml (i.e. a 1:1000 dilution of the stock solution) corresponds to a maximum action for all the blockers. The analysis by immunolabeling using antibodies directed against the SMN protein (commercial and bespoke antibodies) shows that this protein occasionally localizes at the level of CBs in 3% to 6% of nontreated cells or cells treated with DMSO alone (control excipient).

In order to determine whether the calcium channel blockers exerted a positive effect, the inventors counted the cells which had the SMN protein located in the CBs out of 100 cells randomly selected for each experiment.

This experiment was repeated three times. The results obtained from the mean of the first three experiments revealed that the molecules tested significantly increase the number of CBs containing the SMN protein after 24 hours of treatment at 2 micrograms/ml (chi-2, p<0.001, n=300, 100 cells per experiment).

A value of induction by a factor equal to 1 was arbitrarily assigned to DMSO. The results show that the four molecules exerted a significant induction by a factor equal to at least 1.7.

More specifically, the inventors also show that pregabalin exerts a significant induction by a factor equal to 1.7 (chi-2, 0.001<p<0.01, n=2300 cells). Amiodarone hydrochloride exerts a significant induction of approximately 2.8. Gabapentin exerts a significant induction of approximately 3.3. Finally, flunarizine hydrochloride exerts a significant induction of approximately 4.8.

The inventors then verified that these molecules still exerted the same beneficial effect after 78 hours of treatment with a single initial dose.

The statistical analysis from these experiments made it possible to conclude that these molecules were capable of significantly increasing the distribution of the SMN protein in the CBs in the immortalized SMA fibroblast line (chi-2, p<0.0001, n>3000 cells). The analysis by immunolabeling of the SMN protein and the coilin protein (another CBs marker detected using a commercial antibody) confirmed that these two proteins were indeed colocalized in the CBs in the cells treated with each of the four molecules.

The inventors thus succeeded in showing that these four compounds are particularly relevant in the treatment of motor neuronopathies.

2. Verification of the Effects Exerted on Fibroblasts from SMA Patients

The distribution of the SMN protein to Cajal bodies in a series of primary cultures of fibroblasts from the skin of patients suffering from the severe (type I), intermediate (type II) and moderate (type III) form of the disease induced by the action of the calcium channel blockers of the invention was analyzed. The mean values calculated from 3 to 7 experiments (n=300 to 700 cells). The chi-2 value is indicated in table 1.

In order to confirm the effect of the calcium channel inhibitors of the invention, the inventors analyzed the distribution of the SMN protein to Cajal bodies in a series of primary cultures of fibroblasts from the skin of patients suffering from the severe (type I), intermediate (type II) and moderate (type III) form of the disease, induced by the action of the calcium channel blockers of the invention.

These fibroblasts are obtained from:
2 individuals suffering from the severe (type I) form,
2 individuals suffering from the intermediate (type II) form, and
1 individual suffering from the moderate (type III) form.
   The statistical analysis of the results using the chi-2 test is summarized in table 1.

TABLE 1
Statistical analysis, using the chi-2 test, of the action of the
calcium channel blockers of the invention on the distribution
of the SMN protein in CBs in primary cultures of fibroblasts from
the skin of patients suffering from the severe (type I), intermediate
(type II), and moderate (type III) form of the disease

| | Type I | | Type II | | Type III |
|---|---|---|---|---|---|
| | Patient | Patient | Patient | Patient | Patient |
| Compound | 1 | 2 | 3 | 4 | 5 |
| Flunarizine | 2.87 | 27.44 | 6.09 | 10.99 | 12.38 |
| dihydrochloride | | | | | |
| Gabapentin | 5.38 | 36.85 | 3.1 | 0.78 | 5.85 |
| Amiodarone | 0.46 | 7.16 | 0 | 1.2 | 2.72 |
| hydrochloride | | | | | |

The action of the calcium channel blockers of the invention is weaker in the primary cultures of SMA fibroblasts. This is explained by a slower cell metabolism of the primary cultures, whereas the immortalized line divides more rapidly.

The detailed analysis reveals that the two type I cultures do not react in the same way. One of the cultures (patient 2) responds positively to the compounds of the invention on the type I immortalized cells, whereas the second (patient 1) responds significantly only to the gabapentin molecule (chi-2 test, 0.02<p<0.05). The latter is the GM03813 line obtained from Coriell Cell Repositories (USA); it is widely used by the scientific community for carrying out studies on the action of molecules in the SMA field.

The type II and III cultures respond overall more weakly than the positive type I culture. This difference is linked to the number of CBs with the SMN protein at the beginning of the treatment, this number already being higher in the II and III types than in the I types (Renvoisé B, Khoobarry K, Gendron M C, Cibert C, Viollet L and Lefebvre S. Distinct domains of the spinal muscular atrophy protein SMN are required for targeting to Cajal bodies in mammalian cells. Journal of Cell Sciences (2006) 119: 680-692).

Overall, this study shows that the calcium channel blockers of the invention are relevant for the treatment of motor neuronopathies, in particular SMA. However, it appears that flunarizine dihydrochloride is the compound which exerts a positive action for the largest number of primary cultures from SMA patients, all three types included.

In order to evaluate the relationship between the concentration (dose) and the effect (response) on the CBs of the immortalized SMA cells, the inventors constructed a dose-response curve for each of the molecules. The median effective concentration (EC5O) under the experimental conditions described here is approximately 80±22 nM for flunarizine dihydrochloride. The results are listed in table 2.

TABLE 2
Action of the compounds of the invention of the distribution
of the SMN protein in CBs in the immortalized line of fibroblasts
from a patient suffering from the severe (type I) form

| | Concen- | | | | |
|---|---|---|---|---|---|
| | tration at | Maxi- | Dilu- | | Induc- |
| | 2 μg/ml | mum | tion at | EC50, | tion at |
| Compound | (μM) | induction | EC50 | nM | EC50 |
| DMSO | 1:1000 | 1 ± 0.3 | — | — | — |
| Flunarizine | 4.2 | 4.8 ± 1.5 | 1/53 | 80 ± 22 | 2.9 |
| dihydrochloride | | | | | |
| Gabapentin | 11.6 | 4.4 ± 1.0 | 1/55 | 210 ± 38 | 2.9 |
| Amiodarone | 3.0 | 2.8 ± 1.0 | 1/50 | 60 ± 26 | 1.8 |
| hydrochloride | | | | | |

Several published reports indicate that the mean effective dose of flunarizine dihydrochloride administered orally in humans is 10 mg per day, which corresponds to flunarizine dihydrochloride maximum plasma level values of approximately 81±16 nanograms/ml (i.e. approximately 170±34 nM) in a time of between 2 and 4 hours, and the equilibrium state is reached in 5 to 6 weeks. Furthermore, the tissue distribution is considerable and the elimination half-life is long. The plasma concentrations are thus in a concentration range compatible with the concentrations associated with the effects of flunarizine dihydrochloride in our cell model.

The SMN protein is part of a broad multiprotein complex, the most well-studied role of which is the assembly of splicing ribonucleoprotein particles, snRNPs (small nuclear ribonucleoproteins). A defect in this assembly is visualized by a marked decrease in the localization of snRNPs at the level of the CBs in the fibroblasts from SMA patients (Renvoisé B, Khoobarry K, Gendron M C, Cibert C, Viollet L and Lefebvre S. Distinct domains of the spinal muscular atrophy protein SMN are required for targeting to Cajal bodies in mammalian cells. Journal of Cell Sciences (2006) 119: 680-692). In another cell model, it has been published that the presence and the number of CBs is directly linked to the efficiency with which the snRNPs are assembled (Klingauf M., Stanek, D. and Neugebauer, K. M. (2006) Enhancement of U4/U6 small nuclear ribonucleoprotein particle association in Cajal bodies predicted by mathematical modeling. Mol. Biol. Cell., 2006, 17, 4972-4981).

Co-immunolabeling experiments similar to the previous ones were carried out on the immortalized SMA cells using two commercial antibodies, one specific for the coilin protein (CBs marker) and the other for the trimethylguanosine (TMG) cap of the small nuclear RNAs of snRNPs.

These tests show an increase in the SMN protein and in the snRNPs in Cajal bodies in the immortalized SMA line from the severe form of the disease (type I), induced by the calcium channel blockers of the invention. The mean value for the snRNPs is calculated from the three experiments (n=300 cells). The analysis of these immunolabelings shows a significant increase in the snRNPs at the level of the CBs in the cells treated for 24 hours with flunarizine dihydrochloride (chi-2, 0.001<p<0.01), on the basis of three independent experiments on 100 cells per experiment.

This study shows that flunarizine dihydrochloride is the compound which has most successfully exerted a beneficial effect both on:
the accumulation of the SMN protein in the CBs; and
the accumulation of the snRNPs in the CBs.

In conclusion, this study places:
calcium channel blockers of the phenylalkylamine family, in particular flunarizine and salts thereof,
calcium channel blockers of the amino acid family, and
calcium channel blockers of the benzofuran family,
   in a therapeutic strategy for diseases involving motor neuron degeneration, in particular spinal muscular atrophy, amyotrophic lateral sclerosis, primary lateral sclerosis, Kennedy's disease and Charcot-Marie-Tooth disease.

### 3. Effect of Synergy Between Flunarizine Dihydrochloride and Amiodarone Hydrochloride on the Distribution of the SMN Protein to CBs

The inventors tested the effect of the combination of flunarizine dihydrochloride and amiodarone hydrochloride on immortalized SMA fibroblasts isolated from a child suffering from a severe form of the disease, in accordance with the protocols described in examples 1 and 2.

To do this, they observed the effect on the immortalized SMA fibroblasts:
of DMSO (control for which a value of induction by a factor equal to 1 was arbitrarily assigned),
of flunarizine dihydrochloride alone,
of amiodarone hydrochloride alone, and
of the combination of flunarizine dihydrochloride and amiodarone hydrochloride.

It should be noted that the flunarizine dihydrochloride and the amiodarone hydrochloride are used in a 1/1000 dilution compared with examples 1 and 2.

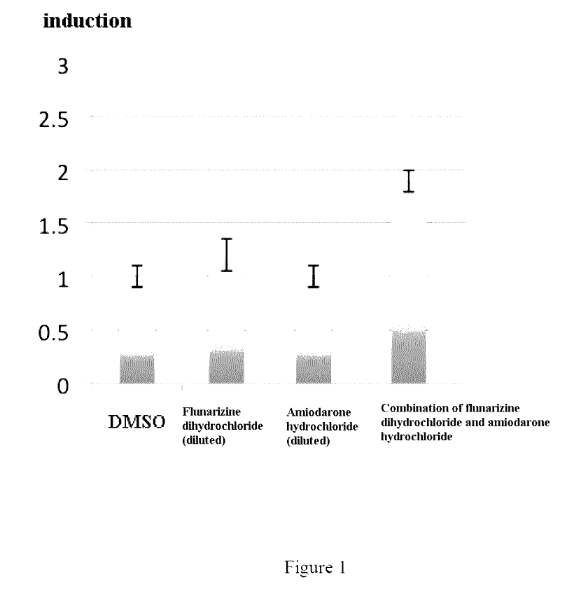

The results are represented in FIG. 1. FIG. 1 shows that flunarizine dihydrochloride and amiodarone hydrochloride act in synergy on the distribution of the SMN protein to CBs.

More specifically, the inventors show that:
flunarizine dihydrochloride alone, diluted to 1/1000, exerts a significant induction by a factor equal to 1.2 (chi-2, 0.001<p<0.01, n=1072 cells);
amiodarone hydrochloride alone, diluted to 1/1000, exerts a non-significant induction by a factor equal to 1 (chi-2, 0.001<p<0.01, n=625 cells);
flunarizine dihydrochloride and amiodarone hydrochloride together exert a significant induction by a factor equal to 1.9 (chi-2, p<0.001, n=2625 cells).

This example shows the surprising and advantageous effect of the combination of flunarizine dihydrochloride and amiodarone hydrochloride. The inventors thus developed a therapeutic strategy particularly relevant for the treatment of motor neuronopathies.

1. A calcium channel blocker selected from the group consisting:
of calcium channel blockers of the phenylalkylamine family of formula I and salts thereof, 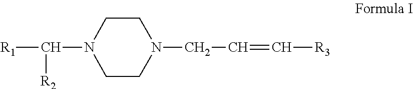
in which:
the R₁, R₂ and R₃ groups are identical or different and are selected from phenyl and fluorophenyl radicals, provided that at least one of the R₁, R₂ and R₃ groups is a fluorophenyl radical;
of calcium channel blockers of the amino acid family of formula II,
H₂N—CH₂—C(R₄)R′₄—CH₂—COOR₅ Formula II
in which:
the R₄ group is a linear- or branched-chain lower alkyl radical containing up to eight carbon atoms and the R′₄ group is a hydrogen atom,
or
R₄ and R′₄ together form a cyclic lower alkyl radical containing up to eight carbon atoms, and
the R₅ group is a hydrogen atom or a linear- or branched-chain lower alkyl radical containing up to eight carbon atoms;
of calcium channel blockers of the benzofuran family of formula III and salts thereof, 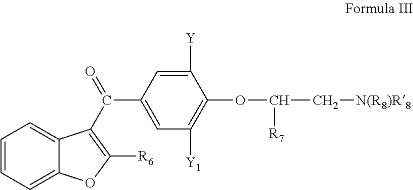
in which:
the R₆ group represents a linear- or branched-chain lower alkyl radical containing up to six carbon atoms,
the R₇ group represents a hydrogen atom or a methyl group,
the N(R₈)R′₈ group represents a dimethylamino, diethylamino, dipropylamino, piperidino, pyrrolidino or morpholino group, and
the Y and Y₁ groups are identical and represent a hydrogen, iodine or bromine atom;
for use thereof in the treatment of a motor neuronopathy selected from spinal muscular atrophy, amyotrophic lateral sclerosis, primary lateral sclerosis, Kennedy's disease and Charcot-Marie-Tooth disease.

2. The calcium channel blocker for use thereof as claimed in claim 1, characterized in that said motor neuronopathy is spinal muscular atrophy.

3. The calcium channel blocker for use thereof as claimed in claim 1, characterized in that said calcium channel blocker belongs to the phenylalkylamine family of formula I or a salt thereof,
in which:
the R₁ and R₂ groups are parafluorophenyl radicals, and
the R₃ group is a phenyl radical.

4. The calcium channel blocker for use thereof as claimed in claim 1, characterized in that said calcium channel blocker belongs to the amino acid family of formula II, in which:
R₄ and R′₄ together form a cyclohexane group, and
the R₅ group is a hydrogen atom.

5. The calcium channel blocker for use thereof as claimed in claim 1, characterized in that said calcium channel blocker belongs to the amino acid family,
in which:
the R₄ group is an isobutyl, and
the R′₄ and R₅ groups are a hydrogen atom.

6. The calcium channel blocker for use thereof as claimed in claim 1, characterized in that said calcium channel blocker belongs to the benzofuran family of formula III, in which:
the R₆ group is a butyl,
the R₇ group is a hydrogen atom,
the N(R₈)R′₈ group is a diethylamino group, and
the Y and Y₁ groups are identical and are an iodine atom.

7. A calcium channel blocker selected from the group consisting of flunarizine and salts thereof, gabapentin, pregabalin, amiodarone and salts thereof, and mixtures thereof, for use thereof in the treatment of the motor neuronopathy selected from spinal muscular atrophy, amyotrophic lateral sclerosis, primary lateral sclerosis, Kennedy's disease and Charcot-Marie-Tooth disease.

8. The calcium channel blocker for use thereof as claimed in claim 7, said calcium channel blocker being flunarizine or a salt thereof.

9. The calcium channel blocker for use thereof as claimed in claim 7, said calcium channel blocker being flunarizine dihydrochloride.

10. The calcium channel blocker for use thereof as claimed in claim 9, characterized in that said calcium channel blocker is administered at a dose of between 5 mg and 10 mg per day.

11. The calcium channel blocker for use thereof as claimed in any one of claims 1 to 9, characterized in that it allows an increase in the distribution of the SMN protein and of snRNPs to Cajal bodies and/or an increase in the number of Cajal bodies.

12. A composition comprising:
a calcium channel blocker of the phenylalkylamine family of formula I and salts thereof, 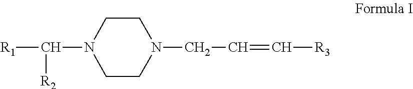
in which:
the R₁, R₂ and R₃ groups are identical or different and are selected from phenyl and fluorophenyl radicals, provided that at least one of the R₁, R₂ and R₃ groups is a fluorophenyl radical;
and
a calcium channel blocker of the benzofuran family of formula III and salts thereof, 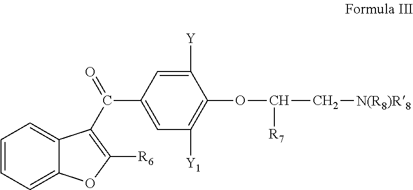
in which:
the R₆ group represents a linear- or branched-chain lower alkyl radical containing up to six carbon atoms,
the R₇ group represents a hydrogen atom or a methyl group,
the N(R₈)R′₈ group represents a dimethylamino, diethylamino, dipropylamino, piperidino, pyrrolidino or morpholino group, and
the Y and Y₁ groups are identical and represent a hydrogen, iodine or bromine atom;
for use thereof in the treatment of a motor neuronopathy selected from spinal muscular atrophy, amyotrophic lateral sclerosis, primary lateral sclerosis, Kennedy's disease and Charcot-Marie-Tooth disease.

13. The composition as claimed in claim 12, characterized in that said calcium channel blocker of the phenylalkylamine family of formula I is flunarizine dihydrochloride and in that said calcium channel blocker of the benzofuran family of formula III is amiodarone hydrochloride.

14. The composition as claimed in claim 12 or 13, characterized in that it is administered at a dose of less than 5 mg per day.

15. A pharmaceutical composition comprising at least one calcium channel blocker as claimed in any of claims 1 to 9, and at least one active agent selected from riluzole, hydroxyurea, ubiquitin/proteasome inhibitors, histone deacetylase inhibitors, protein phosphatase inhibitors, olesoxime, aminoglycosides, salbutamol, isoindoline, ibudilast, kinase modulators, IGF-1 variants, human growth factors, PTK-inhibiting bicyclic pyrimidines, EGFR-inhibiting quinazoline, tyrosine kinase inhibitors, HSP90 inhibitors, myostatin antagonists, low-molecular-weight heparin, neramexane, inhibitors of the protein kinase G3SKB, inhibitors of RhoA and of Rho-associated kinase (ROCK pathway), antioxidants, apoptosis inhibitors and ERK and PI3K/AKT kinase pathway modulators.
